(19) United States
(12) Patent Application Publication
(10) Pub. No.: US 2010/0298769 A1
(43) Pub. Date: Nov. 25, 2010

(54) IMPLANTABLE MEDICAL DEVICES FOR THERAPEUTIC AGENT DELIVERY

(75) Inventors: Scott Schewe, Eden Prairie, MN (US); Robert W. Warner, Woodbury, MN (US); J. Thomas Ippoliti, St. Paul, MN (US); Hollie Beckord, Minneapolis, MN (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/783,024

(22) Filed: May 19, 2010

Related U.S. Application Data
(60) Provisional application No. 61/180,293, filed on May 21, 2009.

(51) Int. Cl. A61M 29/00; A61F 2/00; A61K 47/36; A61P 9/00

(52) U.S. Cl. 604/96.01; 424/422; 514/777

(57) ABSTRACT

Various aspects of the invention relate to implantable medical devices, which comprise a layer of material (e.g., in the form of a sheet, tube, etc.) that comprises a bioerodible polymer and a therapeutic agent. Other aspects of the invention relate to methods of forming such devices. Still other aspects of the invention relate to methods of treatment using such devices.

### RELATED APPLICATIONS

This application claims priority from U.S. provisional application 61/180,293, filed May 21, 2009, which is incorporated by reference herein in its entirety.

### TECHNICAL FIELD

This invention relates to medical devices for therapeutic agent delivery, and more particularly, to medical devices containing biodegradable polymer layers for therapeutic agent delivery.

### BACKGROUND OF THE INVENTION

The in-situ delivery of therapeutic agents within the body of a patient is common in the practice of modern medicine. In-situ delivery of therapeutic agents is often implemented using medical devices that may be temporarily or permanently placed at a target site within the body. These medical devices can be maintained, as required, at their target sites for short or prolonged periods of time, in order to deliver therapeutic agents to the target site.

For example, in recent years, drug eluting coronary stents, which are commercially available from Boston Scientific Corp. (TAXUS), Johnson & Johnson (CYPHER) and others, have been widely used for maintaining vessel patency after balloon angioplasty. These products are based on metallic expandable stents with biostable polymer coatings that release antirestenotic drugs at a controlled rate and total dose.

Therapeutic agents have also been delivered to vessel walls using balloons. For example, recent clinical trials have shown that in-stent restenosis can be treated using a balloon having a sprayed coating of a mixture of paclitaxel and iopromide. B. Scheller et al., Eurointervention Supplement (2008) Vol. 4 (Supplement C)C63-C66.

### SUMMARY OF THE INVENTION

Various aspects of the invention relate to medical devices having at least one bioerodible layer that comprises at least one biodegradable polymer and at least one therapeutic agent.

In some embodiments, the bioerodible layer comprises one or more glycosaminoglycans, which can be optionally crosslinked.

In some embodiments, the bioerodible layer is in the form of a fibrous scaffold, for example, in order to promote three-dimensional migration and proliferation of cells within the scaffold.

In some embodiments, a release material is disposed on at least one surface of the bioerodible layer, which release material promotes release of the bioerodible layer from a delivery device. In some embodiments, an adhesive material is disposed on at least one surface of the bioerodible layer, which adhesive material promotes adhesion of the material to bodily tissue. In some embodiments, a release material is disposed on at least one surface of the bioerodible layer, which release material promotes release from a delivery device, and an adhesive material is disposed at an opposing surface of the bioerodible layer, which adhesive material promotes adhesion of the material to bodily tissue.

Other aspects of the invention relate to methods of forming such devices. For example, in some embodiments, the bioerodible layer is in the form of a fibrous tubular scaffold that is electrospun onto a delivery balloon.

Still other aspects of the invention relate to methods of treatment using such devices. For instance, in some embodiments, a medical device described herein is applied de novo to a plaque lesion in a blood vessel. In some embodiments, a medical device described herein is applied to a previously stented region of a blood vessel.

These and other aspects and embodiments of the present invention will become immediately apparent to those of ordinary skill in the art upon review of the Detailed Description and claims to follow.

### BRIEF DESCRIPTION OF THE DRAWINGS

FIG. 1 is a schematic illustration of a medical device in accordance with an embodiment of the invention.

FIGS. 1A-1C schematically illustrate three alternative cross-sections for the device of FIG. 1, in accordance with various embodiments of the invention.

FIG. 2 is a schematic illustration of a medical device in accordance with another embodiment of the invention.

FIGS. 2A-2C schematically illustrate three alternative cross-sections for the device of FIG. 2, in accordance with various embodiments of the invention.

FIG. 3 is a schematic longitudinal partial cross-sectional view illustrating a balloon catheter with an associated balloon-deliverable device, in accordance with an embodiment of the invention.

FIG. 4 is a schematic partial cross-sectional view illustrating the use of the balloon catheter of FIG. 3 for deploying the balloon-deliverable device in the vasculature, in accordance with an embodiment of the invention.

FIG. 5 is a schematic partial cross-sectional view illustrating the balloon-deliverable device of FIG. 3, after deployment in the vasculature and removal of the balloon catheter, in accordance with an embodiment of the invention.

### DETAILED DESCRIPTION

As previously indicated, various aspects of the invention relate to medical devices having at least one bioerodible layer (e.g., in the form of a sheet, tube, etc.) that comprises at least one biodegradable polymer and at least one therapeutic agent. Such a layer is referred to herein as a “bioerodible polymer-containing layer”.

The medical devices of the present invention include a variety of implantable and insertable medical devices that are used for the treatment of various mammalian tissues and organs. As used herein, “treatment” refers to the prevention of a disease or condition, the reduction or elimination of symptoms associated with a disease or condition, or the substantial or complete elimination of a disease or condition. Subjects are vertebrate subjects, more typically mammalian subjects including human subjects, pets and livestock.

Examples of medical devices benefiting from the present invention vary widely and include implantable or insertable medical devices, for example, stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, catheters (e.g., urological catheters or vascular catheters such as balloon catheters and various central venous catheters), guide wires, balloons, filters (e.g., vena cava filters and mesh filters for distil protection devices), embolization devices including cerebral aneurysm filler coils (including Guglielmi detachable coils and metal coils), septal defect closure devices, myocardial plugs, patches, electrical stimulation leads, including leads for pacemakers, leads for implantable cardioverter-defibrillators, leads for spinal cord stimulation systems, leads for deep brain stimulation systems, leads for peripheral nerve stimulation systems, leads for cochlear implants and leads for retinal implants, ventricular assist devices including left ventricular assist hearts and pumps, total artificial hearts, shunts, valves including heart valves and vascular valves, anastomosis clips and rings, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, orthopedic prosthesis such as bone grafts, bone plates, fins and fusion devices, joint prostheses, orthopedic fixation devices such as interference screws in the ankle, knee, and hand areas, tacks for ligament attachment and meniscal repair, rods and pins for fracture fixation, screws and plates for craniomaxillofacial repair, dental implants, or other devices that are implanted or inserted into the body and from which therapeutic agent is released.

In certain embodiments the medical devices of the invention include patches and drug-delivery sleeves, which may or may not be associated with a structural member such as a stent.

As used herein “layer” of a given material is a region of that material whose thickness is substantially less that its length and width (e.g., its length and width are each at least five times as great as its thickness, frequently much greater). Layers can be in the form of open structures (e.g., sheets, in which case the thickness of the layer is substantially less than the length and width of the layer), partially closed structures (e.g., open tubes, in which case the thickness of the layer is substantially less than the length and diameter of tube) and fully closed structures (e.g., spheres and closed tubes, in which case the thickness of the layer is substantially less than the length and/or diameter of the structure).

As used herein, a polymer is “biodegradable” if it undergoes bond cleavage along the polymer backbone in vivo, regardless of the mechanism of bond cleavage (e.g., enzymatic breakdown, hydrolysis, oxidation, etc.). “Bioerosion” or “bioabsorption” of a polymer-containing component of a medical device (e.g., a polymer-containing layer) is defined herein to be a result of polymer biodegradation (as well as other in vivo disintegration processes such as dissolution, etc.) and is characterized by a substantial loss in vivo over time (e.g., the period that the device is designed to reside in a patient) of the original polymer mass of the component. For example, losses may range from 50% to 75% to 90% to 95% to 97% to 99% or more of the original polymer mass of the device component. Bioabsorption times may vary widely, with typical bioabsorption times ranging from several hours to approximately one year.

As discussed in more detail below, in various embodiments, bioerodible polymer-containing layers in accordance with the invention may be in the form of a fibrous scaffold with an open porous structure that encourages three-dimensional migration and proliferation of cells within the fibrous scaffold.

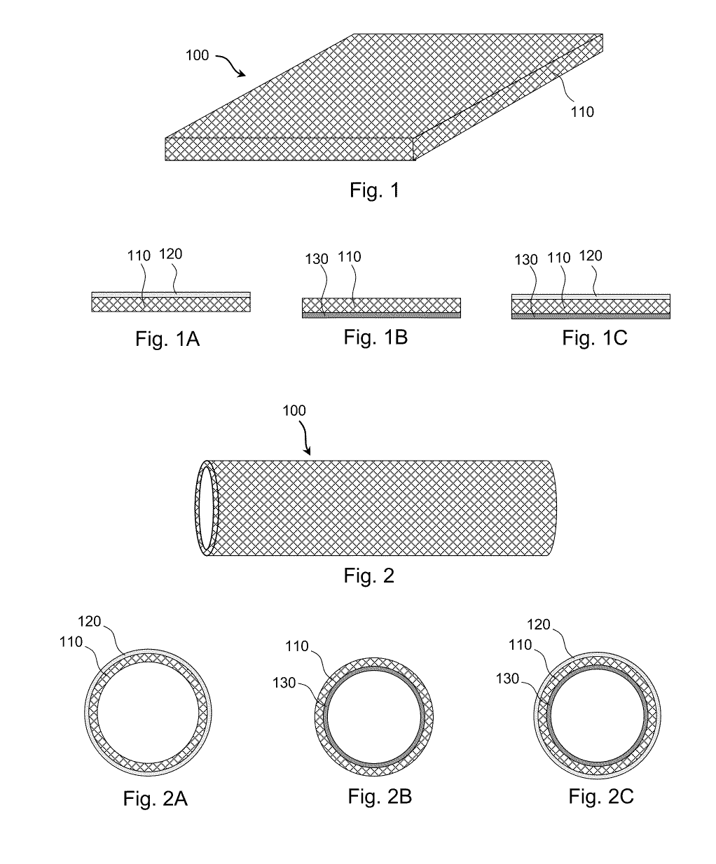

FIG. 1 is a schematic perspective view of a medical device 100 in accordance with an embodiment of the present invention. The device 100 is in the form of a bioerodible polymer-containing layer, specifically a sheet 110 (e.g., a drug delivery patch). As discussed more fully below and as seen from the end views of FIGS. 1A-1C, in certain embodiments, an adhesive layer 120 may be formed on one surface of the sheet 110 (FIG. 1A), a release layer 130 may be formed on one surface of the sheet 110 (FIG. 1B), or an adhesive layer 120 may be formed on one surface of the sheet 110 and a release layer 130 may be formed on an opposing surface of the sheet 110 (FIG. 1C).

FIG. 2 is a schematic perspective view of a medical device 100 in accordance with another embodiment of the present invention, which is in the form of a bioerodible polymer-containing layer, specifically, a tube 110 (e.g., a sleeve for vascular implantation). As discussed more fully below and as seen from the end views of FIGS. 2A-2C, in certain embodiments, an adhesive layer 120 may be formed on an outer surface of the tube 110 (FIG. 2A), a release layer 130 may be formed on an inner surface of the tube 110 (FIG. 2B), or an adhesive layer 120 may be formed on an outer surface of the tube 110 while a release layer 130 may be formed on an inner surface of the tube 110 (FIG. 2C).

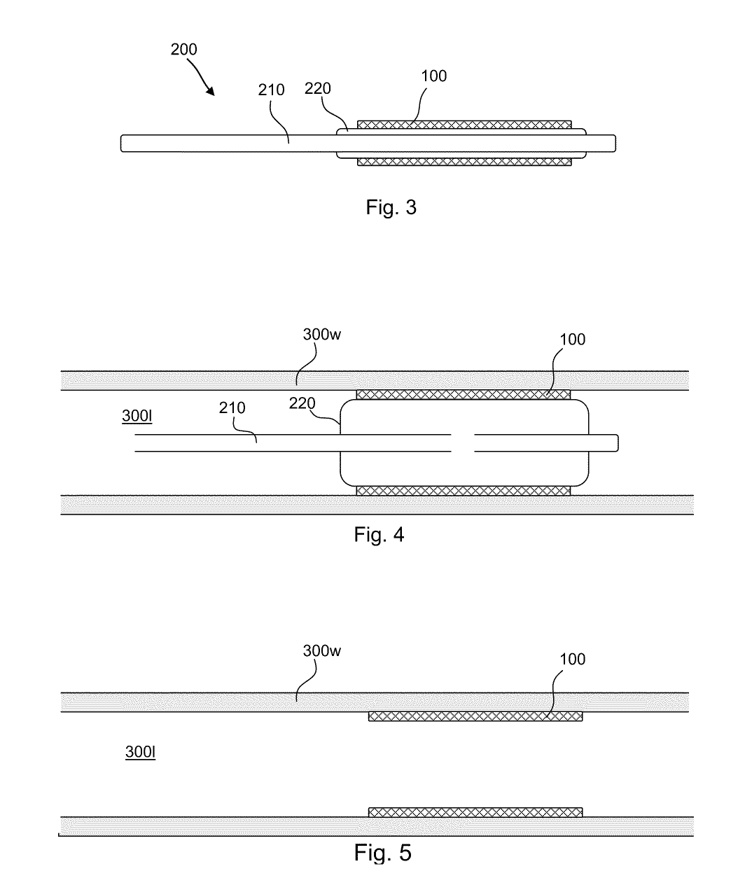

Such devices 100 may be delivered to the body using a suitable delivery device. For example, turning to FIG. 3, there is shown a schematic cross-section of a balloon catheter 200 which is adapted for insertion into a blood vessel lumen. The catheter 200 includes a balloon 220 disposed on a catheter body 210. A device 100 like that of FIG. 2C is provided on the surface of the balloon. Accordingly, although not separately shown, an adhesive layer is provided on an outer surface of the device and a release layer is provided on an inner surface of the device.

Turning now to FIG. 4, the catheter 200 of FIG. 3 may be inserted into a blood vessel lumen 3001. As the balloon 220 is inflated, the device 100 is expanded (e.g., unfolded along with the balloon) into contact with the blood vessel wall 300w. The adhesive layer on the outer surface of the device 100 enhances adhesion between the device 100 and the vessel wall 300w upon contact, as described in more detail below. In addition the release layer on the inner surface of the device 100 enhances release of the device 100 from the balloon 220, as described in more detail below. Upon removal of the catheter 200 from the site, the device 100 remains adhered to the vessel wall 300w as shown if FIG. 5.

Bioerodible polymer-containing layers for use in the invention typically contain, for example, from 1 to 100 wt % of one or more biodegradable polymers, more preferably, from 25 to 50 to 75 to 90 to 95 to 99 wt % or more of one or more biodegradable polymers.

Bioerodible polymer-containing layers for use in the invention may vary, for example, from 100 nm to 1 micron(μm) to 10 micron to 50 micron to 100 micron or more in thickness.

Examples of bioerodible polymer-containing layers include non-porous layers and porous layers (e.g., fibrous layers).

Polymers which may be used to form bioerodible polymer-containing layers for use in the invention include synthetic and natural biodegradable polymers. Synthetic biodegradable polymers include polyesters, for example, selected from homopolymers and copolymers of lactide, glycolide, and epsilon-caprolactone, including poly(l-lactide), poly(d,l-lactide), poly(lactide-co-glycolides) such as poly(l-lactide-co-glycolide) and poly(d,l-lactide-co-glycolide), polycarbonates including trimethylene carbonate (and its alkyl derivatives), polyphosphazines, polyanhydrides and polyorthoesters. Natural biodegradable polymers include proteins, for example, selected from fibrin, fibrinogen, collagen and elastin, and polysaccharides, for example, selected from chitosan, gelatin, starch, and glycosaminoglycans such as chondroitin sulfate, dermatan sulfate, keratan sulfate, heparin, heparan sulfate, and hyaluronic acid. Blends of the above natural and synthetic polymers may also be employed.

Various preferred embodiments are discussed below in which the biodegradable polymer is a glycosaminoglycan such as hyaluronic acid (also called hyaluronan or hyaluronate) and/or heparin, although it is clear that other bioerodible polymers, including other glycosaminoglycans, may be employed.

Hyaluronic acid (HA) is a polymer of disaccharides composed of D-glucuronic acid and D-N-acetylglucosamine, linked together via alternating β-1,4 and β-1,3 glycosidic bonds. It is a non-sulfated glycosaminoglycan distributed widely throughout connective, epithelial, and neural tissues. It is one of the chief components of the extracellular matrix and contributes significantly to cell proliferation and migration, including that of endothelial cells. HA also possesses several pharmacological properties including inhibition of platelet adhesion and aggregation, and stimulation of angiogenesis. HA has been successfully used in bioactive agent delivery applications. See Samir Ibrahima et al., “A surface-tethered model to assess size-specific effects of hyaluronan (HA) on endothelial cells,” Biomaterials 28 (2007) 825-835. HA varies widely in molecular weight, typically ranging from 8×10² or less to 8×10⁶ or more, more typically ranging from 5×10³ to 8×10⁶ in the present invention.

In certain embodiments, the HA in the bioerodible polymer-containing layers of the invention is crosslinked. Crosslinking reduces the solubility of the HA and may also reduce the release rate of any therapeutic disposed within the HA-containing layers. Thus, therapeutic agent release kinetics may be controlled by adjusting the degree of crosslinking within the HA component.

HA may be crosslinked, for example, using water-soluble carbodiimide. See A. Sannino et al., Polymer, 46(25), 2005, 11206-11212. HA has also been crosslinked using glutaraldehyde, poly(ethyelene glycol) diglycidyl ether, 1-ethyl-3-(3-dimethylaminopropyl) carbodiimide (EDC) or divinyl sulfone (DVS) as crosslinking agents. M. N. Collins et al., Journal of Applied Polymer Science, 104(5), 2007, 3183-3191. See further Y. Ji et al., Biomaterials 27 (2006) 3782-3792 who describe the crosslinking of 3,3′-dithiobis(propanoic dihydrazide)-modified HA through poly(ethylene glycol)-diacrylate.

In certain embodiments, a naturally occurring biodegradable cross-linking agent is used. One example of such a cross-linking agent is genipin. Genipin is a hydrolytic product of geniposide, which is found in the fruit of Gardenia jasminoides Ellis. Because it is a naturally occurring, biodegradable molecule with low cytotoxicity, genipin has recently been investigated as a crosslinking material in various applications.

Genipin may also provide anti-inflammatory effects and also potentially anti-thrombus effects. Hye-Jin Koo et al., “Anti-inflammatory evaluation of gardenia extract, geniposide and genipin,” Journal of Ethnopharmacology, 103(3), 2006, 496-500, Y. Suzuki et al., “Antithrombotic effect of geniposide and genipin in the mouse thrombosis model,” Planta medica, 67(9), 2001, 807-810. As noted above, HA may also have therapeutic affects (see Samir Ibrahima et al., supra), which along with genipin may contribute to a synergistic treatment of tissue, including diseased blood vessels.

Like HA, heparin is an extended polymer of repeating sugar units. It is widely used as an anticoagulant. As the chemical structure between HA and heparin are similar, the effect on modification with crosslinking will be similar to HA. In some embodiments, heparin may be the only biodegradable polymer in the bioerodible polymer-containing layer. In some embodiments, HA may be utilized as the as the primary biodegradable polymer with smaller amounts of heparin provided for its anti-thrombus properties. For example, in certain embodiments, the biodegradable polymer content of the polymer layer may comprise from 1 to 100 wt % HA and from 1 to 100 wt % heparin as bioerodible polymers. If desired, heparin may be crosslinked, for example, using agents such as those described above for HA.

As noted above, examples of bioerodible polymer-containing layers include non-porous layers (e.g., hydrogel layers) and porous layers (e.g., fibrous layers). Non-porous layers may be provided using techniques such as by dipping, spray coating, coating with an applicator (e.g., by roller, brush, etc), and so forth.

Fibrous layers may be formed using, for example, fiber spinning techniques. For example, electrospinning is a fiber spinning technique by which a suspended drop of polymer (e.g., a polymer in a suitable solvent) is charged with tens of thousands of volts. At a characteristic voltage the droplet forms a Taylor cone, and a fine jet of polymer releases from the surface in response to the tensile forces generated by interaction of an applied electric field with the electrical charge carried by the jet. This produces a filament of material. This jet can be directed to a grounded surface such as a balloon delivery system and collected as a continuous web of fibers that can be adjusted to give fibers ranging in size, for example, from 50 nm to 100 nm to 250 nm to 500 nm to 1 micron to 2.5 microns to 5 microns to 10 microns to 20 microns. To ensure good coverage, the balloon delivery system may be rotated and reciprocated relative to the jet. Multiple dispensers with differing concentrations of starting materials may be utilized to produce higher concentrations of selected materials in specific areas of the nanofibrous network. Further information on electrospinning may be found, for example, in US 2005/0187605 to Greenhalgh et al. See also Y. Ji et al., “Electrospun three-dimensional hyaluronic acid nanofibrous scaffolds,” Biomaterials 27 (2006) 3782-3792.

Porous layers including electrospun fibrous layers increase available surface area and therefore may increase release of any therapeutic agents and increase biodegradation rate relative to nonporous layers. Moreover, such layers may serve to create a scaffold for cell seeding, growth and/or proliferation. For example, in the case of vascular devices, such layers may serve as a scaffold for endothelial cell seeding, growth and/or proliferation in vivo.

In various embodiments, a crosslinking agent may be included, for example, along with one or more biodegradable polymers in a solution that is used to form the bioerodible polymer-containing layer (assuming a suitable crosslinking agent is selected that is not so fast acting so as to hinder layer formation). As an alternative, a crosslinking agent and a biodegradable polymer may be simultaneously deposited on a surface (e.g., from separate containers) to form a bioerodible polymer-containing layer. As another alternative, a crosslinking agent may be applied to a biodegradable polymer layer after it is formed.

In various embodiments, one or more therapeutic agents may also be included, for example, along with one or more biodegradable polymers in a solution that is used to form a bioerodible polymer-containing layer. As an alternative, a biodegradable polymer and one or more therapeutic agents may be simultaneously deposited (e.g., from separate containers) to form a bioerodible polymer-containing layer. As another alternative, one or more therapeutic agents may be applied (e.g., in solution) to the bioerodible polymer-containing layer after it is formed.

A wide variety of therapeutic agents may be used in the devices of the invention. Numerous therapeutic agents are described below.

In various embodiments, the bioerodible polymer-containing layer is in the form of a tubular sleeve that is delivered to the vasculature for treatment of coronary artery disease or treatment of in-stent restenosis. For instance, the invention may employ a balloon-based system for delivery.

In some embodiments, a tubular sleeve in accordance with the invention can be used to deliver therapeutic agents to de novo lesion sites. In other embodiments, a tubular sleeve in accordance with the invention can be used to deliver therapeutic agents to the site of a previously deployed stent. In still other embodiments, a stent may be coadministered along with one or more tubular sleeves in accordance with the invention (e.g., the sleeve may be disposed on an abluminal surface of the stent, the luminal surface of the stent, or both).

Examples of therapeutic agents for these embodiments include anti-plaque agents, agents that promote endothelial layer formation, and anti-restenotic agents (e.g., to prevent restenosis due to vessel injury, to address existing in-stent restenosis), among others. Examples of antirestenotic agents include taxanes such as estradiol, genistein, paclitaxel and olimus family drugs, among many others. Examples of agents that promote endothelial layer formation include endothelial progenitor cells (EPC) and growth factors such as VEGF, among many others. Examples of anti-plaque agents include lipid-lowering drugs such as statins, ACE inhibitors, beta blockers, antioxidants, macrolide antibiotics and anti-inflammatory agents, including inhibitors of MMP, among many others. Additional therapeutic agents are described below.

In one specific example, a tubular sleeve in accordance with the invention is disposed over a standard angioplasty balloon (e.g., formed directly on the balloon or formed and then disposed on the balloon). Such a tubular sleeve represents a stent-like configuration that is released from the delivery device after being fully dilated and opposed into the lesion site. The sleeve typically facilitates a controlled release of a biologically active agent (e.g., paclitaxel, olimus family drugs, etc.) and in some embodiments, selectively adheres to the diseased portion of the vessel, for example, to facilitate active agent uptake.

In systems incorporating a fibrous tubular scaffold disposed on a high pressure dilatation balloon, the act of deploying the balloon may embed the fibrous material into the plaque lesion material, exposing the fiber surfaces to the lesion for elution of the active agents. The fact that the fiber is embedded into the lesion may lessen or eliminate the need for lesion selective adhesion strategies.

Nonetheless, in certain aspects of the invention, various strategies are employed to facilitate adhesion of a device in accordance with the invention (e.g., a tubular sleeve or patch) to the wall of a body lumen. In many embodiments, strategies are employed to facilitate adhesion to a blood vessel, and in some instances to a plaque lesion in a blood vessel (e.g., a coronary artery, etc.).

For example, in some embodiments, one or more adhesive substances can be provided in the bioerodible polymer-containing layer (e.g., evenly dispersed in the layer or, more preferably, having a higher concentration at a tissue contacting surface of the layer). In some embodiments, one or more adhesive substances can be provided in an adhesive layer that is disposed over the surface of the bioerodible polymer-containing layer (which adhesive layer may penetrate the bioerodible polymer-containing layer to a certain degree). For example, a pure layer of an adhesive substance or a layer containing an adhesive substance and a suitable adjuvant may be applied to a tissue contacting surface of a bioerodible polymer-containing layer in accordance with the invention. Several examples of adhesive substances are discussed in the following paragraphs.

Because plaque lesions are known to be hydrophobic, a hydrophobic drug (e.g., paclitaxel, among many others) may be provided over or within the bioerodible polymer-containing layer, encouraging adhesion and/or uptake by the lesion upon contact with a lesion.

In other embodiments, a polar molecule may be employed as an adhesive substance. Examples of such polar molecules include poly(amino acids). For instance, in some embodiments, an amphipathic poly(amino acid) is used as an adhesive substance. The amphipathic poly(amino acid) may have a hydrophobic poly(amino acid) tail (e.g., ranging from 2 to 400 or more amino acids in length) to encourage interaction with the lesion. Examples of hydrophobic amino acids include phenylalanine, leucine, isoleucine and valine, among others. The amphipathic poly(amino acid) may have a hydrophilic poly(amino acid) head (e.g., ranging from 2 to 400 or more amino acids in length) to encourage interaction with the biodegradable polymer (where a hydrophilic polymer such as HA is employed). Examples of hydrophilic amino acids include basic amino acids (e.g., lysine, arginine, histidine, ornithine, etc.), acidic amino acids (e.g., glutamic acid, aspartic acid, etc.), and neutral amino acids (e.g., cysteine, asparagine, glutamine, serine, threonine, tyrosine, glycine).

In certain embodiments, the hydrophilic poly(amino acid) head is zwitterionic to promote ion-dipole bonding with the biodegradable polymer (where a hydrophilic polymer such as HA is employed). Such a polymer head will contain a mixture of acidic (anionic) and basic (cationic) amino acids and may range, for example, from 2 to 400 or more amino acids in length.

In other embodiments, a poly(amino acid) which contains a cell-binding peptide such as YIGSR or RGD is employed as an adhesive substance. Such sequences can be repeated if desired. The poly(amino acid) may further comprise a hydrophilic poly(amino acid) chain (e.g., typically ranging from 2 to 400 or more amino acids in length) to promote interaction with the bioerodible polymer (where a hydrophilic polymer such as HA is employed).

In other embodiments, the amino acid 3,4 dihydroxyphenyl alanine (DOPA) or a poly(amino acid) chain that comprises multiple DOPA units is used as an adhesive substance. Such chains may further include lysine units, along with the DOPA units. See Statz et al. J. Am. Chem. Soc. 127, 2005, 7972-7973, wherein a 5-mer anchoring peptide (DOPA-Lys-DOPA-Lys-DOPA) was chosen to mimic the DOPA- and Lys-rich sequence of a known mussel adhesive protein.

In still other embodiments, MSCRAMMs (microbial surface components recognizing adhesive matrix molecules) are employed as adhesive substances. Examples of MSCRAMMs include fibronectin binding proteins (e.g., FnBPA, FnBPB, etc.) and fibrinogen binding proteins (e.g., C1fA, C1fB, etc.), among others. See, e.g., Timothy J. Foster, Chapter 1, “Surface protein adhesins of staphylococci,” from Bacterial Adhesion to Host Tissues: Mechanisms and Consequences, Edited by Michael Wilson, 2002, pages 3-11

In certain aspects of the invention, various strategies are employed to facilitate release of a device in accordance with the invention (e.g., a sleeve, patch, etc.) from a delivery vehicle (e.g., from the balloon of a balloon catheter).

Examples of balloon materials include relatively non-complaint materials such as polyamides, for instance, polyamide homopolymers and copolymers and composite materials in which a matrix polymer material, such as polyamide, is combined with a fiber network (e.g., Kevlar® an aramid fiber made by Dupont or Dyneema®, a super-strong polyethylene fiber made by DSM Geleen, the Netherlands). Specific examples of polyamides include nylons, such as nylon 6, nylon 4/6, nylon 6/6, nylon 6/10, nylon 6/12, nylon 11 and nylon 12 and poly(ether-co-amide) copolymers, for instance, polyether-polyamide block copolymer such as poly(tetramethylene oxide-b-polyamide-12) block copolymer, available from Elf Atochem as PEBAX. Examples of balloon materials also include relatively complaint materials such as silicone, polyurethane or compliant grades of PEBAX having a larger percentage of polyether, for example PEBAX 63D.

For example, in certain embodiments, devices in accordance with the invention are bound to delivery vehicles using substances whose binding capability can be disrupted (referred to herein as “release substances”).

For instance, in some embodiments, one or more release substances can be provided in the bioerodible polymer-containing layer (e.g., evenly dispersed in the layer or more preferably having a higher concentration at a delivery vehicle contacting surface of the layer). In some embodiments, one or more release substances can be provided in a release layer that is disposed between the surfaces the delivery vehicle and the bioerodible polymer-containing layer (which release layer may penetrate the bioerodible polymer-containing layer to a certain degree).

One example of a release substance is zwitterionic phosphorylcholine,

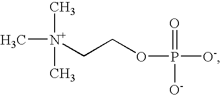

which has also been demonstrated as an anti-thrombus material in the medical device arena and has been used for this purpose on drug eluting stents. Phosphorylcholine is able to form ionic-dipole bonds with various polar substances, including bioerodible polymers such as HA and polar balloon materials such as PEBAX. In this way phosphorylcholine may act to bind the bioerodible polymer portion of the sleeve to the balloon material. When desired, a wetting agent (e.g., saline or water) can be employed to disrupt the ionic-dipole interactions holding the sleeve on the balloon.

In some embodiments, the wetting agent is supplied by the delivery vehicle. For instance, an inflatable micro-porous or weeping balloon may be used to dilate the vessel site and deliver a wetting agent which interacts with the zwitterionic phosphorylcholine. As another example, saline loaded microspheres may be provided between the bioerodible polymer-containing layer and the balloon, which burst and release their contents upon balloon inflation.

Derivatives of phosphorylcholine may also be employed. For example, amphiphilic phosphorylcholine derivatives with non-polar tails such as dipalmitoylphosphatidyl choline (DPPC), i.e.,

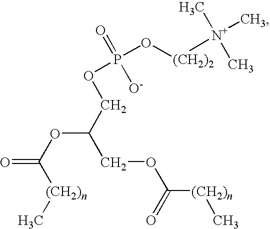

where n is 14 or 1-O-octadecyl-2-O-methyl-sn-glycero-3-phosphorylcholine,

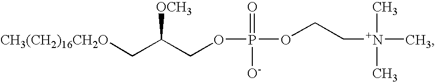

may be used for binding devices in accordance with the invention to hydrophobic balloon materials. In these embodiments, the polar phosphorylcholine head portion is employed to form ionic-dipole bonds polar bioerodible polymers such as HA, while the hydrophobic alkyl portions of these molecules are employed to interact with an adjacent nonpolar balloon material such as nylon or polyurethane, thereby binding the bioerodible polymer portion of the sleeve to the balloon material. When desired, a wetting agent (e.g., saline or water) can be employed to disrupt the ionic-dipole interactions between the zwitterionic portion of the phosphorylcholine derivative and the hydrophilic bioerodible polymer portion of the sleeve.

Similarly, other zwitterionic materials may be employed as release substances including zwitterionic peptides. For example, peptides with both basic amino acids (e.g., lysine, arginine, ornithine, etc.) and acidic amino acids (e.g., glutamic acid, aspartic acid, etc.) will have zwitterionic character for providing ionic ionic-dipole bonds with various polar substances (e.g., a hydrophilic bioerodible polymer or a hydrophilic balloon material). Chains of non-polar amino acid chains (e.g., phenylalanine, leucine, isoleucine, valine, etc.) may be attached to zwitterionic chains for providing hydrophobic interactions with various nonpolar substances (e.g., a hydrophobic balloon material).

Shear sensitive adhesives constitute another class of release substance that may be used between a balloon delivery vehicle and a device in accordance with the invention. The basic principle of these adhesives is that the shearing force that is created between the inflating balloon and the adhesive will break the bond and facilitate release. An example of such an adhesive is a blend of polyvinylpyrrolidone (PVP) and polyethylene glycol (PEG), which would provide a biocompatible layer which adheres the balloon to the bioerodible polymer-containing layer until the device is in place at the delivery site. Balloon dilation may be used to disrupt the adhesive bonds and the bioerodible polymer-containing layer may thus be released from the balloon. The weight ratio of PVP to PEG in such blends may vary widely, for example, ranging from 1:99 to 10:90 to 25:75 to 50:50 to 75:25 to 90:10 to 95:5 to 99:1.

Where the delivery device is a balloon, the device may be applied to the balloon in a folded state to minimize interactions between the device and the balloon that would have to be disrupted for device delivery, thereby improving release.

In some embodiments, devices in accordance with the invention are created and then applied to a delivery device. For example, a drug delivery sleeve comprising an inner release layer, a drug-releasing bioerodible fibrous layer, and an outer adhesive layer may be formed and applied to a balloon, which may be folded in certain embodiments. Optionally, a stent may be provided (a) before application of the sleeve (in the event an abluminal fibrous layer is desired for the stent) or (b) after application of the fibrous layer (in the event a luminal fibrous layer is desired for the stent). As another example, a drug delivery sleeve comprising an inner release layer, a first drug-releasing bioerodible fibrous layer, a stent, a second drug-releasing bioerodible fibrous layer, and an outer adhesive layer may be formed and applied to a balloon, which may be folded in certain embodiments. Different drugs may be supplied in the fibrous layers, for example, an endothelial cell growth promoter may be provided in the inner/lumenal fibrous layer and an antirestenotic drug may be provided in the outer/ablumenal fibrous layer.

In other embodiments, devices in accordance with the invention may be formed on the surface of the delivery device. As a specific example (among many other possibilities), a release layer may first be applied to a surface of an inflatable balloon. A fibrous bioerodible polymer-containing layer and a therapeutic agent is then formed over the release layer. In a subsequent step, an adhesive layer is provided over the fibrous bioerodible polymer-containing layer. As a more specific example, a release layer may first be applied to a surface of an inflatable balloon formed from a material such as nylon, polyurethane or PEBAX, among others. The release layer may comprise, among other possibilities, (a) a shear sensitive adhesive or (b) a zwitterionic release substance such as phosphorylcholine in combination with saline microcapsules (unless a micro-porous or weeping balloon is employed, in which case the saline microcapsules will be excluded). A fibrous layer, for example, comprising HA and paclitaxel as a therapeutic agent is then formed over the release layer, for instance, using an electrospinning process. The HA in the fibrous layer may then be crosslinked by applying genipin to the fibrous layer. In a subsequent step, DOPA is applied to the outer fiber layer surface as an adhesive substance, among other possibilities.

Optionally, a stent may be provided (a) before application of the fibrous layer (in the event an abluminal fibrous layer is desired), (b) after application of the fibrous layer (in the event a luminal fibrous layer is desired) or (c) after application of one fibrous layer, followed by formation of another fibrous layer (in the event that a fiber encapsulated stent structure with luminal and abluminal fibrous layers is desired).

“Therapeutic agents,” “pharmaceutically active agents,” “pharmaceutically active materials,” “drugs,” “biologically active agents” and other related terms may be used interchangeably herein and include genetic therapeutic agents, non-genetic therapeutic agents and cells. A wide variety of therapeutic agents can be employed in conjunction with the present invention including those used for the treatment of a wide variety of diseases and conditions.

Exemplary therapeutic agents for use in connection with the present invention include: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists, including PPAR-alpha, gamma and delta agonists, such as rosiglitazone, pioglitazone, netoglitazone, fenofibrate, bexaotene, metaglidasen, rivoglitazone and tesaglitazar, (bb) prostaglandin E agonists, including PGE2 agonists, such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, (ff) phospholipids including phosphorylcholine, phosphatidylinositol and phosphatidylcholine, (gg) VLA-4 antagonists and VCAM-1 antagonists.

Specific therapeutic agents include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, biolimus, zotarolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Numerous therapeutic agents, not necessarily exclusive of those listed above, have been identified as candidates for vascular treatment regimens, for example, as agents targeting restenosis (antirestenotics). Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, 5-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid, SOD (orgotein) and SOD mimics, verteporfin, rostaporfin, AGI 1067, and M 40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat, metastat, batimastat, pentosan polysulfate, rebimastat, incyclinide, apratastat, PG 116800, RO 1130830 or ABT 518, (y) cell motility inhibitors such as cytochalasin B, (z) antiproliferative/antineoplastic agents including antimetabolites such as purine antagonists/analogs (e.g., 6-mercaptopurine and pro-drugs of 6-mercaptopurine such as azathioprine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, biolimus, zotarolimus, etc.), cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, (cc) blood rheology modulators such as pentoxifylline and (dd) glucose cross-link breakers such as alagebrium chloride (ALT-711).

Numerous additional therapeutic agents useful for the practice of the present invention are also disclosed in U.S. Pat. No. 5,733,925 to Kunz, the entire disclosure of which is incorporated by reference.

### EXAMPLE

An initial layer of phosphorylcholine (PC) is formed up to one micron in thickness as a sleeve release agent on a delivery vehicle (e.g., a folded, PEBAX delivery balloon) by either spraying or dipping the delivery vehicle in a solution of PC dissolved in a first solvent such as THF (tetrahydrofuran) or diethyl ether. Once the first solvent has sufficiently dried, a drug-loaded hyaluronic acid layer (HA/drug layer) is formed on the PC coated delivery vehicle to between 0.5 and 5 microns in thickness by spraying or dipping the delivery vehicle in a solution of HA and a therapeutic agent such as paclitaxel dissolved in a second solvent such as THF or DMF (dimethylformamide). The second solvent is evaporated leaving the HA/drug layer over the PC layer. A final tissue-adhesive layer is then applied in a similar fashion, by spraying or dipping the delivery vehicle in a solution of DOPA and HA dissolved in a third solvent such as THF or DMF, thereby forming a DOPA/HA layer of up to 1 micron in thickness. This is followed by drying to evaporate the third solvent leaving the DOPA/HA layer over the HA/drug layer, which is in turn disposed over the PC layer.

Although various embodiments are specifically illustrated and described herein, it will be appreciated that modifications and variations of the present invention are covered by the above teachings and are within the purview of the appended claims without departing from the spirit and intended scope of the invention.

1. An implantable medical device comprising a fibrous tubular scaffold that comprises crosslinked hyaluronic acid and a therapeutic agent.

2. The implantable medical device of claim 1, further comprising a release layer on an inner surface of the fibrous tubular scaffold that promotes release from a delivery device.

3. The implantable medical device of claim 1, further comprising an adhesive layer on an outer surface of the fibrous tubular scaffold that promotes adhesion of the fibrous scaffold to bodily tissue.

4. The implantable medical device of claim 3, wherein said adhesive layer promotes adhesion of the fibrous scaffold to a blood vessel wall.

5. The implantable medical device of claim 1, wherein the fibrous tubular scaffold further comprises heparin.

6. The implantable medical device of claim 1, wherein the fibrous tubular scaffold is crosslinked with a biodegradable cross-linking agent.

7. The implantable medical device of claim 1, wherein said therapeutic agent is selected from anti-plaque agents, anti-restenotic agents, and endothelium promoting agents.

8. The implantable medical device of claim 1, wherein the device further comprises a stent.

9. A delivery system comprising a balloon catheter and a fibrous tubular scaffold that comprises a bioerodible polymer and a therapeutic agent, wherein said fibrous tubular scaffold is electrospun onto the balloon of a balloon catheter.

10. The delivery system of claim 9, wherein said fibrous tubular scaffold comprises a glycosaminoglycan.

11. The delivery system of claim 9, wherein said fibrous tubular scaffold comprises hyaluronic acid.

12. The delivery system of claim 9, wherein said fibrous tubular scaffold comprises hyaluronic acid and heparin.

13. The delivery system of claim 9, wherein a layer of material that promotes release from the balloon is applied to the balloon prior to electrospinning the fibrous scaffold onto the balloon.

14. The delivery system of claim 9, wherein a layer of material that promotes adhesion of the fibrous tubular scaffold to bodily tissue is applied on an outer surface of the fibrous tubular scaffold.

15. An implantable medical device comprising (a) a scaffold in the form of a sheet or a tube that comprises a bioerodible polymer and a therapeutic agent, (b) a release material on one surface of the fibrous scaffold that promotes release from a delivery device and (c) a adhesive material on an opposing surface of said fibrous scaffold that promotes adhesion of said fibrous scaffold to bodily tissue.

16. The implantable medical device of claim 15, wherein said scaffold comprises crosslinked hyaluronic acid and a therapeutic agent.

17. The implantable medical device of claim 15, wherein said release material comprises a zwitterionic molecule.

18. The implantable medical device of claim 17, wherein said zwitterionic molecule is selected from phosphorylcholine, phosphorylcholine derivatives that comprise one or more alkyl chains, and amphipathic peptide amino acids comprising a hydrophobic polyamino acid portion and a zwitterionic hydrophilic polyamino acid portion.

19. The implantable medical device of claim 17, further comprising saline containing microcapsules.

20. The implantable medical device of claim 15, wherein said release material comprises a shear sensitive adhesive.

21. The implantable medical device of claim 20, wherein said shear sensitive adhesive is a blend of polyvinylpyrrolidone (PVP) and polyethylene glycol (PEG).

22. The implantable medical device of claim 15, wherein said adhesive material comprises a hydrophobic drug.

23. The implantable medical device of claim 15, wherein said adhesive material comprises an MSCRAMM.

24. The implantable medical device of claim 15, wherein said adhesive material comprises an amphipathic peptide amino acid comprising a hydrophobic polyamino acid portion and a hydrophilic polyamino acid portion.

25. The implantable medical device of claim 15, wherein said adhesive material comprises 3,4 dihydroxyphenyl alanine (DOPA).

26. The implantable medical device of claim 15, wherein said adhesive material is a poly(amino acid) that comprises 3,4 dihydroxyphenyl alanine (DOPA).

27. The implantable medical device of claim 15, wherein said therapeutic agent selected from wherein said therapeutic agent is selected from anti-plaque agents, anti-restenotic agents and endothelium promoting agents.

28. A method of treatment comprising applying the device of claim 1 to a blood vessel.

29. A method of treatment comprising applying the device of claim 15 to a blood vessel.

30. A method of treatment comprising inserting the delivery system of claim 9 into a blood vessel and inflating said balloon.
